(19) United States
(12) Patent Application Publication
(10) Pub. No.: US 2019/0392926 A1
(43) Pub. Date: Dec. 26, 2019

(54) METHODS AND SYSTEMS FOR PROVIDING AND ORGANIZING MEDICAL INFORMATION

(71) Applicant: 5 Health Inc., Palo Alto, CA (US)

(72) Inventors: Dorothea Li Feng KOH, Waltham, MA (US); Yan Chuan SIM, San Francisco, CA (US)

(21) Appl. No.: 16/016,330

(22) Filed: Jun. 22, 2018

(51) Int. Cl. G16H 10/60; H04L 12/58; G06F 17/28; G10L 15/08; G06F 3/0482; G16H 80/00

(52) U.S. Cl. CPC G16H 10/60; H04L 51/02; H04L 51/10; H04L 51/08; G10L 2015/088; G10L 15/08; G06F 3/0482; G16H 80/00; G06F 17/28

(57) ABSTRACT

A method for processing information includes causing display of a user interface on a user computing device, wherein the user interface comprises a conversation simulator, receiving user input from at least one user provided through the user interface, the user input relating to medical treatment of a patient, predicting a user intent based on at least one keyword in the user input, determining medical content based on the user intent and at least one candidate medical content associated with the user intent, automatically generating a response to the user input based on the user intent and medical content, and causing display of the generated response to the user input on the user interface through the conversation simulator.

### TECHNICAL FIELD

This invention relates generally to the field of medical treatment and more specifically to aggregating and providing medical information.

### BACKGROUND

Healthcare professionals (e.g., physicians) may be able to provide more effective medical treatment to patients if they are able to consistently and easily access clinical and/or other medical information. For example, it may be desirable to quickly obtain or verify drug information or patient information from a patient's medical file (e.g., medical images). Conventional medical resources include hard copy printed resources (e.g., books, paper-based patient medical records). Printed resources are not easily or reliably updateable, and information that is no longer accurate may detract from proper medical treatment. Furthermore, there may be limited access to printed resources because they are difficult to share among multiple users. Some other medical resources are digital or electronics-based, but tend to be time-consuming and/or difficult to navigate to obtain desired information, which may lead to unnecessary and harmful delays in providing medical treatment to patients. Even further, various resources (e.g., medical records, medical knowledge databases, etc.) are typically discrete, such that healthcare professionals must separately consult various databases to obtain the information they seek, thereby further complicating the ability to effectively provide medical treatment.

### SUMMARY

In some aspects of the methods and systems described herein, a user may engage in chat conversations within an artificial intelligence environment, such as with an artificial intelligence medical assistant (e.g., represented by a chatbot or other conversation simulator) and/or one or more other users. The artificial intelligence medical assistant may provide medical information to one or more users in response to user inputs (e.g., queries) within a chat conversation. Additionally, media such as images or videos, or other attachments such as links or medical calculators may be shared among users and/or the artificial intelligence medical assistant. Furthermore, a user may create notes (e.g., associated with a patient) such as through text entry and/or dictation. Various medical information in the chats and/or notes may be generated and/or stored in new and/or existing electronic medical records associated with patients.

For example, generally, a method for processing information may include causing display of a user interface on a user computing device, wherein the user interface comprises a conversation simulator, receiving user input from at least one user provided through the user interface, the user input relating to medical treatment of a patient, predicting a user intent based on at least one keyword in the user input, determining medical content based on the user intent and at least one candidate medical content associated with the user intent, automatically generating a response to the user input based on the user intent and medical content; and causing display of the generated response to the user input on the user interface through the conversation simulator.

As another example, generally, a system for processing information may include one or more processors configured to cause display of a user interface on a user computing device, wherein the user interface comprises a conversation simulator, receive user input from at least one user through the user interface, the user input relating to medical treatment of a patient, predict a user intent based on at least one keyword in the user input, determine medical content based on the user intent and at least one candidate medical content associated with the user intent, automatically generate a response to the user input based on the user intent and medical content, and cause display of the generated response to the at least one user on the user interface through the conversation simulator.

The user input to be analyzed by the methods and systems described herein may involve any suitable kind of input, such as text-based user input and/or spoken user input. Such user input may, for example, be provided via a user computing device (e.g., mobile phone, tablet, laptop or desktop computer, etc.).

In some variations, the conversation simulator may be associated with a natural language processing model. The natural language processing model may, for example, predict a user intent by determining at least one synonym of an identified keyword in the user input. The natural language processing model may furthermore, for example, determine medical content by mapping the identified keyword and/or at least one synonym of the keyword to at least one medical content candidate. In some variations, determining medical content may include determining a relevance score for each medical content candidate and comparing the relevance scores. In some variations, at least a portion of medical content may be stored in an electronic medical record associated with the patient.

In some variations, the user input may include dialogue between two or more users within the user interface, and an artificial intelligence system may monitor the dialogue between the two or more users for particular user input warranting response. For example, monitoring dialogue between two or more users may include identifying at least a first keyword associated with user intent and a second keyword associated with medical content. At least a portion of the medical content may be stored in an electronic medical record associated with the patient.

Models for identifying user intent and/or determining medical content may be updated based at least in part on user feedback. For example, after providing a generated response to user input, a user may be prompted to provide feedback on the quality of the generated response. After receiving the user feedback, the model (e.g., natural language processing model) may be modified based at least in part on the user feedback, such as by adjusting weighting factors used to determine relevance scores for medical content candidates.

### BRIEF DESCRIPTION OF THE DRAWINGS

FIG. 1 is a schematic illustration of an exemplary architecture for an artificial intelligence (AI) environment.

FIG. 2 is a schematic illustration of an exemplary variation of a user computing device.

FIG. 3 is a schematic illustration of an exemplary variation of an artificial intelligence medical assistant system.

FIG. 4A is an illustrative flowchart depicting an exemplary interaction between an artificial intelligence medical assistant system and a user computing device.

FIG. 4B is an illustrative flowchart depicting an exemplary variation of a method for predicting user intent and determining medical content in an artificial intelligence environment.

FIG. 4C is an illustrative flowchart depicting an exemplary variation of a method for incorporating feedback to update a model for predicting user intent and determining medical content in an artificial intelligence environment.

FIG. 5 is a schematic illustration of an exemplary variation of an AI environment.

FIG. 6A is an exemplary variation of a GUI displaying a list of chat conversations within the AI environment.

FIG. 6B is an exemplary variation of a GUI displaying options for establishing a new chat conversation.

FIG. 6C is an exemplary variation of a GUI displaying a contact list of other users operating within the AI environment.

FIG. 7A is an exemplary variations of a GUI displaying an individual chat with an AI medical assistant that is associated with a conversation simulator (e.g., chatbot).

FIG. 7B is another exemplary variation of a GUI displaying an individual chat with an AI medical assistant that is associated with a conversation simulator (e.g., chatbot).

FIG. 8A is an exemplary variation of a GUI displaying a medical information box generated by an AI medical assistant within a conversation.

FIG. 8B is an exemplary variation of a GUI displaying a drug interaction information box generated by an AI medical assistant within a conversation.

FIG. 8C is an exemplary variation of a GUI displaying an expanded drug interaction information box generated by an AI medical assistant within a conversation.

FIG. 9A is an exemplary variation of a GUI displaying options for a medical calculator generated by an AI medical assistant within a conversation.

FIG. 9B is an exemplary variation of a GUI showing a medical calculator result after the user has selected a medical calculator.

FIG. 9C is an exemplary variation of a GUI displaying a medical calculator with fields for receiving values entered by a user through the user interface.

FIG. 9D is an exemplary variation of a GUI that illustrates an expanded medical calculator with fields for receiving values entered by a user through the user interface.

FIG. 10A is an exemplary variation of a GUI displaying at least one image within a conversation with the AI medical assistant.

FIG. 10B is an exemplary variation of a GUI displaying at least one video within a conversation with the AI medical assistant.

FIG. 11A is an exemplary variation of a GUI displaying a response by the AI medical assistant in response to a user input within a conversation with one or more other users.

FIG. 11B is an exemplary variation of a GUI displaying file sharing with another user within a conversation in the AI environment.

FIG. 12A is an exemplary variation of a GUI displaying a group chat conversation.

FIG. 12B is an exemplary variation of a GUI displaying options for configuring a group chat conversation.

FIG. 13A is an exemplary variation of a GUI configured to display notes that are accessible to a user.

FIG. 13B is an exemplary variation of a GUI displaying a menu of note-taking options.

FIG. 14A is an exemplary variation of a GUI displaying a freestyle voice note-taking interface.

FIG. 14B is an exemplary variation of a GUI displaying a transcribed freestyle voice note.

FIG. 15 is an exemplary variation of a GUI displaying a dictated voice case note-taking interface.

FIG. 16 is an exemplary variation of a GUI displaying an interface for completing a typed case note.

FIG. 17 is an exemplary variation of a GUI displaying an interface for completing a freestyle typed note.

FIG. 18 is an exemplary variation of a GUI displaying an exemplary note.

### DETAILED DESCRIPTION

Non-limiting examples of various aspects and variations of the invention are described herein and illustrated in the accompanying drawings.

### Overview

Generally, described herein is an artificial intelligence (AI) environment that provides and organizes medical information for a user such as a healthcare professional (e.g., physician, nurse, etc.). In some variations, the AI environment may include an electronic medical record platform and an AI medical assistant system. One or more users may interact with a user interface on a user computing device (e.g., mobile device such as a mobile phone or tablet, or other suitable computing device such as a laptop or desktop computer, etc.) that is in communication with the AI environment. For example, the AI medical assistant system may be configured to interpret and respond to user input such as user queries for medical information in a readily accessible manner through a machine-implemented conversation simulator such as a chatbot. User input may, for example, request information regarding drugs (e.g., drug description, dosage guidelines, drug interactions, etc.), diseases, medical calculators, etc. A user may additionally or alternatively communicate with other users over a network through the user interface, such as to share medical information (e.g., over chat conversations, by sharing files such as images or videos, by sharing links to content, etc.) and/or otherwise collaborate on medical care for a patient. At least some of the medical information relating to a patient may be automatically identified by the AI medical assistant system as suitable for storage in an electronic medical record for the patient and subsequently automatically stored in the electronic medical record. Additionally or alternatively, the user interface may enable a user to contribute medical information to an electronic medical record for a patient such as through verbal and/or audio-based notetaking, or other designation of medical information for storage in an electronic medical record. Accordingly, in some variations the methods and systems described herein may aggregate and provide a wide variety of medical information in a centralized platform, thereby enabling easy and efficient access to medical information (from medical resource databases, from electronic medical records, from other members of a patient care team, etc.) and improving medical care and treatment of patients.

### AI Environment

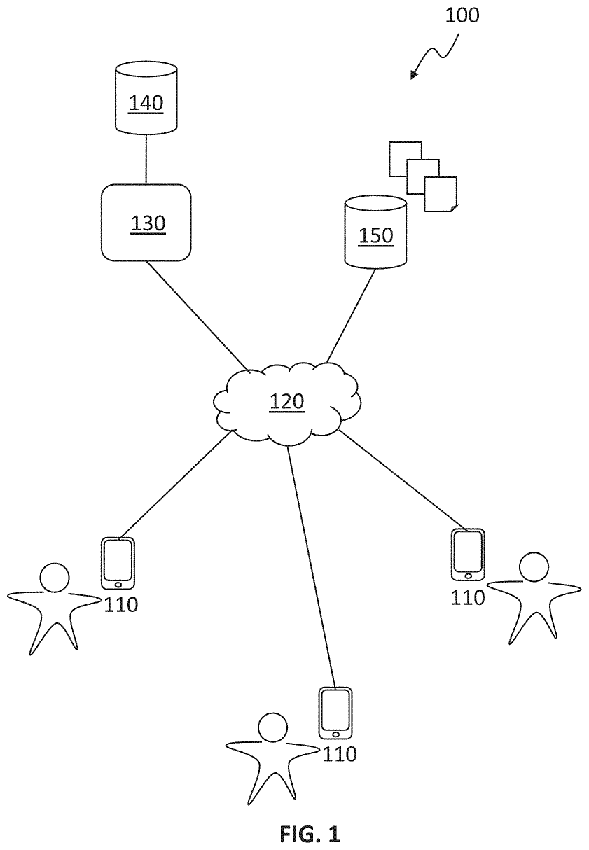

FIG. 1 illustrates an exemplary architecture for an AI environment 100. As shown in FIG. 1, one or more user computing devices 110 are operated by respective users (e.g., physicians, nurse practitioners, nurses, medical assistants, etc.) and may be communicatively connected to a network 120. As described in further detail below, each of the user computing devices may be configured to communicate with other user computing devices 110 within the AI environment 100. An AI medical assistant system 130 may also be communicatively connected to the network 120, as well as one or more medical resource databases 140 that the medical assistant system 130 may access for medical information. One or more of the user computing devices 110 and/or the medical assistant system 130 may be communicatively connected over the network 120 to an electronic medical record (EMR) database configured to store electronic medical records for one or more patients, such that a user computing device 110 and/or the medical assistant system 130 may be configured to read and/or write information to electronic medical records over the network 120.

### Computing Devices

In some variations, a user computing device 110 may include a mobile computing device (e.g., mobile phone, tablet, personal digital assistant, etc.) or other suitable computing device (e.g., laptop computer, desktop computer, other suitable network-enabled device, etc.).

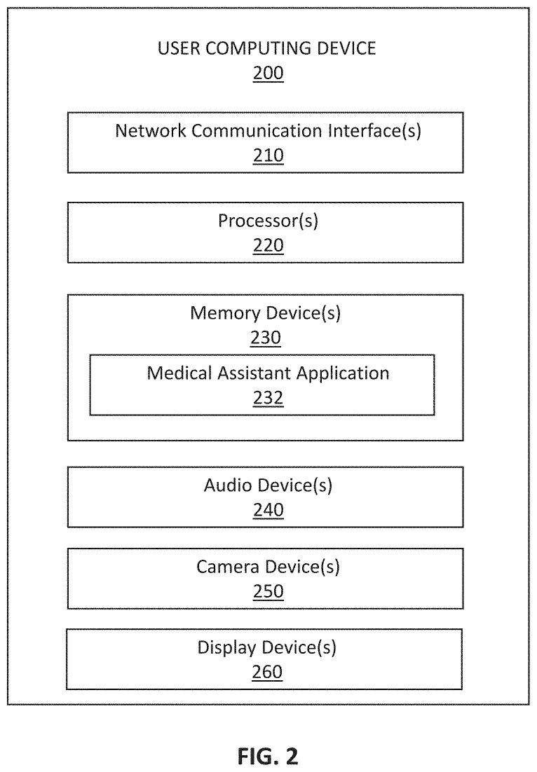

Generally, as shown in FIG. 2 schematically depicting a user computing device 200, the computing devices described herein may include a controller including at least one processor 220 (e.g., CPU) and at least one memory device 230 (which can include one or more computer-readable storage mediums). The processor 220 may incorporate data received from the memory device 230, user input, for example. The memory device 230 may include stored instructions to cause the processor to execute modules, processes, and/or functions associated with the methods described herein. In some variations, the memory device and processor may be implemented on a single chip, while in other variations they can be implanted on separate chips.

The processor 220 may be any suitable processing device configured to run and/or execute a set of instructions or code, and may include one or more data processors, image processors, graphics processing units, physics processing units, digital signal processors, and/or central processing units. The processor may be, for example, a general purpose processor, a Field Programmable Gate Array (FPGA), an Application Specific Integrated Circuit (ASIC), and/or the like. The processor may be configured to run and/or execute application processes and/or other modules, processes and/or functions associated with the system and/or a network associated therewith. The underlying device technologies may be provided in a variety of component types (e.g., MOSFET technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and/or the like.

In some variations, the memory device 230 may include a database and may be, for example, a random access memory (RAM), a memory buffer, a hard drive, an erasable programmable read-only memory (EPROM), an electrically erasable read-only memory (EEPROM), a read-only memory (ROM), Flash memory, and the like. The memory device may store instructions to cause the processor to execute modules, processes, and/or functions such as measurement data processing, measurement device control, communication, and/or device settings. Some variations described herein relate to a computer storage product with a non-transitory computer-readable medium (also may be referred to as a non-transitory processor-readable medium) having instructions or computer code thereon for performing various computer-implemented operations. The computer-readable medium (or processor-readable medium) may be non-transitory in the sense that it does not include transitory propagating signals per se (e.g., a propagating electromagnetic wave carrying information on a transmission medium such as space or a cable). The media and computer code (also may be referred to as code or algorithm) may be those designed and constructed for the specific purpose or purposes.

Examples of non-transitory computer-readable media include, but are not limited to, magnetic storage media such as hard disks, floppy disks, and magnetic tape; optical storage media such as Compact Disc/Digital Video Discs (CD/DVDs); Compact Disc-Read Only Memories (CDROMs), and holographic devices; magneto-optical storage media such as optical disks; solid state storage devices such as a solid state drive (SSD) and a solid state hybrid drive (SSHD); carrier wave signal processing modules; and hardware devices that are specially configured to store and execute program code, such as Application-Specific Integrated Circuits (ASICs), Programmable Logic Devices (PLDs), Read-Only Memory (ROM), and Random-Access Memory (RAM) devices. Other variations described herein relate to a computer program product, which may include, for example, the instructions and/or computer code disclosed herein.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including C, C++, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

In some variations, the memory device 230 may store a medical assistant application 232 configured to enable the computing device 200 operate within the AI environment (e.g., communicate with other computing devices within the AI environment, communicate with a medical assistant system, etc.) as further described herein. The medical assistant application 232 may, for example, be configured to render a text chat interface that facilitates conversation with other users of the medical assistant application 232 on other computing devices, and/or conversation with an AI medical assistant system.

In some variations, a computing device may include at least one communication interface 210 configured to permit a user to control the computing device. The communication interface may include a network interface configured to connect the computing device to another system (e.g., Internet, remote server, database) by wired or wireless connection. In some variations, the computing device may be in communication with other devices via one or more wired or wireless networks. In some variations, the communication interface may include a radiofrequency receiver, transmitter, and/or optical (e.g., infrared) receiver and transmitter configured to communicate with one or more device and/or networks.

Wireless communication may use any of a plurality of communication standards, protocols, and technologies, including but not limited to, Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (WiFi) (e.g., IEEE 802.11a, IEEE 802.11b, IEEE 802.11g, IEEE 802.11n, and the like), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol.

The communication interface 210 may further include a user interface configured to permit a user (e.g., patient, health care professional, etc.) to control the computing device. The communication interface may permit a user to interact with and/or control a computing device directly and/or remotely. For example, a user interface of the computing device may include at least one input device for a user to input commands and/or at least one output device for a user to receive output (e.g., prompts on a display device). Suitable input devices include, for example, a touchscreen to receive tactile inputs (e.g., on a displayed keyboard or on a displayed UI), and a microphone to receive audio inputs (e.g., spoken word).

Suitable output devices include, for example, an audio device 240, a display device 260, and/or other device for communicating with the patient through visual, auditory, tactile, and/or other senses. In some variations, the display may include, for example, at least one of a light emitting diode (LED), liquid crystal display (LCD), electroluminescent display (ELD), plasma display panel (PDP), thin film transistor (TFT), organic light emitting diodes (OLED), electronica paper/e-ink display, laser display, holographic display, or any suitable kind of display device. In some variations, an audio device may include at least one of a speaker, a piezoelectric audio device, magnetostrictive speaker, and/or digital speaker. Other output devices may include, for example, a vibration motor to provide vibrational feedback to the patient. In some variations, the user computing device 200 may include at least one camera device 250, which may include any suitable optical sensor (e.g., configured to capture still images, capture videos, etc.).

### Network

In some variations, the systems and methods described herein may be in communication via, for example, one or more networks, each of which may be any type of wired network or wireless network. A wireless network may refer to any type of digital network that is not connected by cables of any kind. Examples of wireless communication in a wireless network include, but are not limited to, cellular, radio, satellite, and microwave communication. However, a wireless network may connect to a wired network in order to interface with the Internet, other carrier voice and data networks, business networks, and personal networks. A wired network may be carried over copper twisted pair, coaxial cable and/or fiber optic cables. There are many different types of wired networks including wide area networks (WAN), metropolitan area networks (MAN), local area networks (LAN), Internet area networks (IAN), campus area networks (CAN), global area networks (GAN) like the Internet, and virtual private networks (VPN). “Network” may refer to any combination of wireless, wired, public, and private data networks that may be interconnected through the Internet to provide a unified networking and information access system. Furthermore, cellular communication may encompass technologies such as GSM, PCS, CDMA or GPRS, W-CDMA, EDGE or CDMA200, LTE, WiMAX, and 5G networking standards. Some wireless network deployments may combine networks from multiple cellular networks or use a mix of cellular, Wi-Fi, and satellite communication.

### AI Medical Assistant System

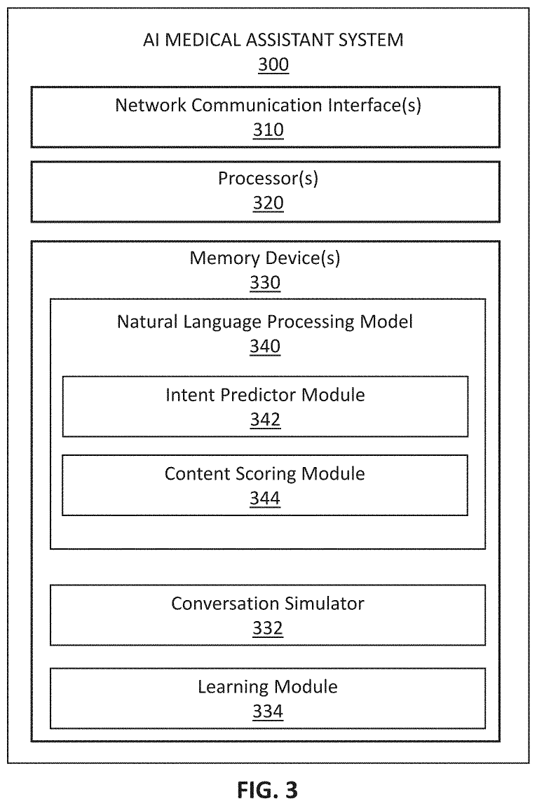

Generally, as shown in the exemplary schematic of FIG. 3, an AI medical assistant system 300 may include at least one network communication interface 310, at least one processor 320, and at least one memory device 330, which may be similar to network communication interface 210, processor 220, and/or memory device 230 described above with respect to FIG. 2. In some variations, one or more servers may host the AI medical assistant system 300 by including the one or more processors 320 and/or one or more memory devices 330.

As shown in FIG. 3, the one or more memory devices 330 may store a natural language processing model 330 (natural language processor) and a conversation simulator 332. The natural language processing model 330 and/or the conversation simulator 332 may be stored on one or multiple memory devices, in any suitable architecture (e.g., distributed, local, etc.). Generally, as further described below, the natural language processing model 330 may be configured to parse user input (e.g., queries or other statements), predict a user intent according to an intent predictor module 342, and attempt to determine suitable medical content associated with the predicted user intent according to a content scoring module 344. The conversation simulator 332 may be configured to emulate human conversation with a user to, for example, communicate information such as medical content in response to user input, or prompt the user for additional information, as further described below. In some variations, the memory device(s) 330 may further include a learning module 334 configured to update and modify the natural language processing model 330 based on supplemental information such as user feedback that characterizes the quality of the medical content provided to the user.

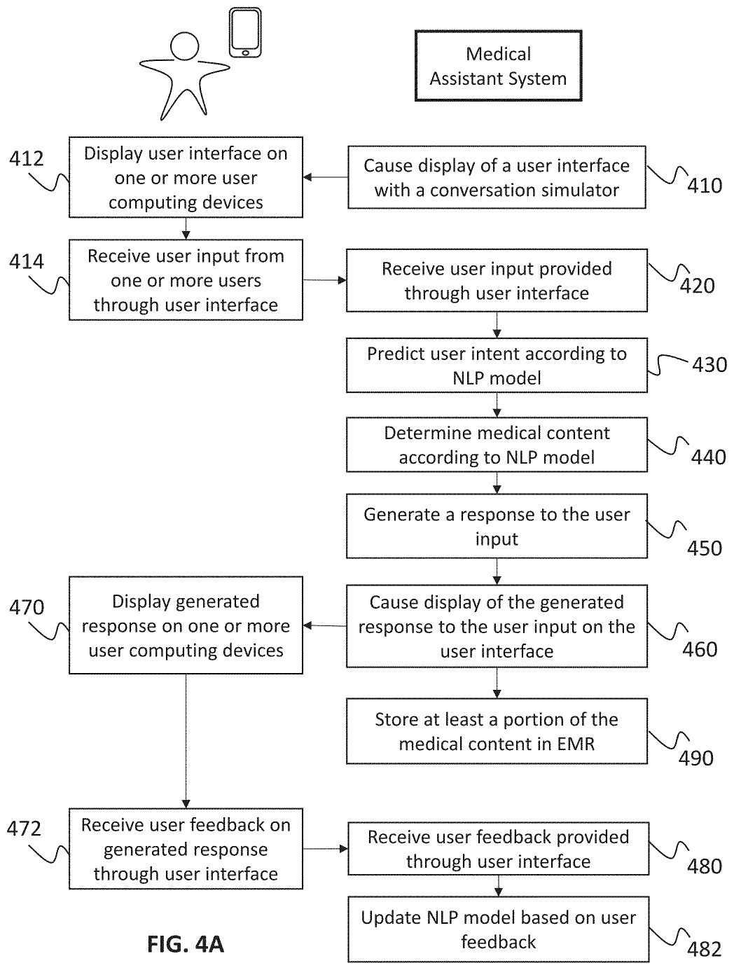

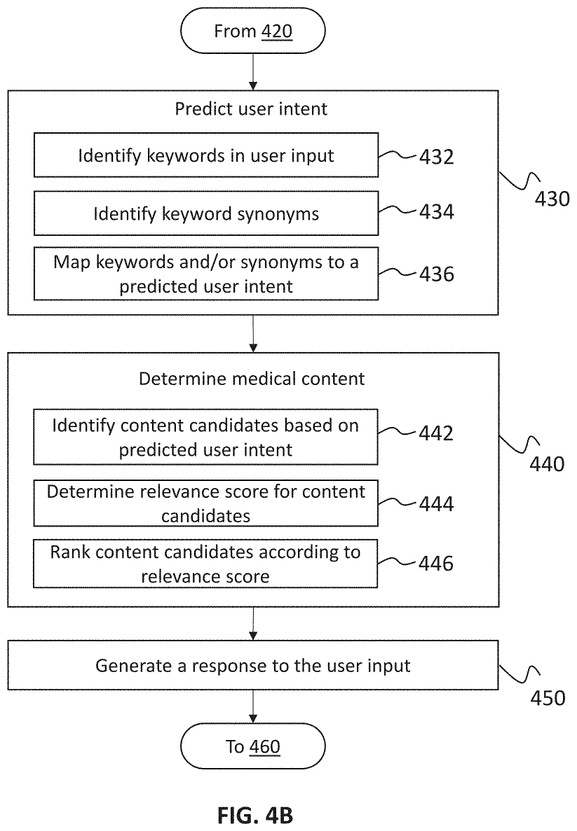

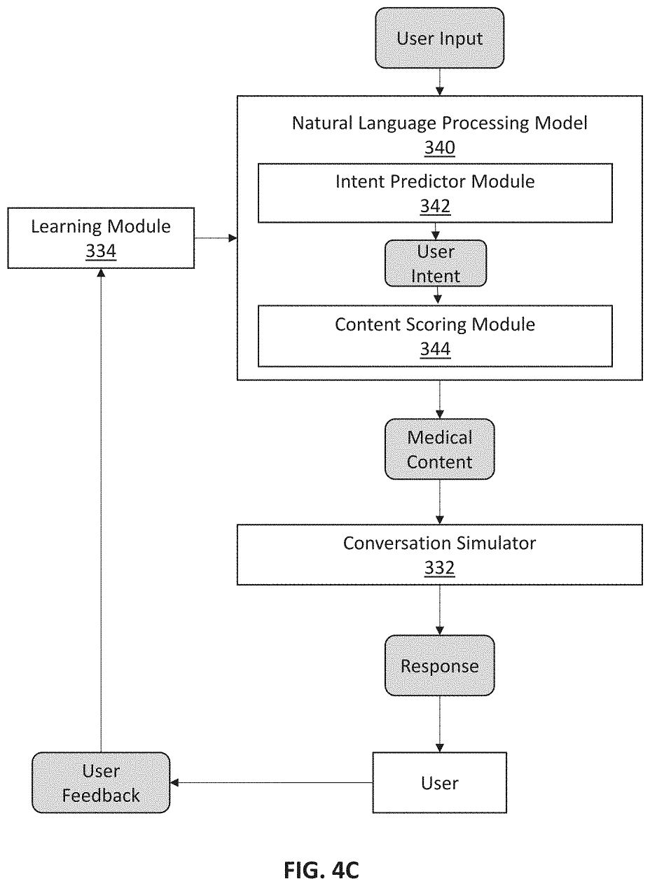

An exemplary interaction between the medical assistant system and a user computing device associated with a user is shown in FIG. 4A. Although the steps and processes of FIG. 4A are ordered in an exemplary sequence, it should be understood that they may alternatively be performed in any suitable order and/or some processes may be performed concurrently.

As shown in FIG. 4A, a medical assistant system (for example, AI medical assistant system 300 described above) may cause display of a user interface with a conversation simulator (410). For example, through a medical assistant application (e.g., loaded on a mobile phone, tablet, etc.) and/or a website interface, a user interface with a conversation simulator may be rendered and displayed on a user computing device (412). For example, the user interface may include a chat interface that may enable text conversations with one or more other users, and/or with the AI medical assistant system. As another example, an interface enabling input of user-entered notes may be displayed on the user computing device. Additional examples of user interfaces are described in further detail below.

User input may be received through the user interface on the user computing device (414) and provided to the medical assistant system. The medical assistant system may receive the user input (420), such as text- or voice-based input. An intent predictor module (e.g., intent predictor module 342) may process the user input to predict user intent (430), and a content scoring module (e.g., content scoring module 344) may determine medical content (440) from the user intent.

FIG. 4B illustrates one exemplary variation of predicting user intent and determining medical content. As shown in FIG. 4B, after receiving user input provided through the user interface (420), the medical assistant system may identify at least one keyword in the user input (432). Keywords may, for example, be identified based on comparing words against a database of known or predetermined words of importance (e.g., “diagnose”, “calculate”, “treat”, medication or drug names, etc.). In some variations, the medical assistant system may be configured to identify one or more synonyms of identified keywords (434), such as by searching a thesaurus or other suitable database that matches or associates keywords with related meanings. The synonyms may, in some variations, be used to expand the range of variety of medical content candidates that may be mapped to the user intent.

The medical assistant system may be configured to map at least a portion of the keywords and/or synonyms of keywords to a predicted user intent (436). For example, the intent predictor module may include or be associated with a natural language processing (NLP) model that is trained to associate a word to a predicted user intent. The NLP model may, for example, incorporate a suitable machine learning model or suitable NLP that is trained on a training dataset including vetted or identified associations between keywords and meanings, and/or user feedback that updates or improves associations between keywords and meanings (e.g., as described in further detail below). Accordingly, the NLP model may be configured to map words such as a keyword in the user input (and/or a synonym of the keyword) to at least one predicted user intent.

One or more potential medical content can identified based at least in part on the predicted user intent (442). Medical content may be identified by matching the predicted user intent to various content in a medical resource database (e.g., medical encyclopedia). For each content candidate, a relevance score or other metric may be determined (444) such as by a content scoring module 344, where the relevance score characterizes the relevance of that content to the predicted user intent. The relevance score may be expressed numerically and on any suitable scale (e.g., 0-100, 0-50, 0-10, etc.), or in any suitable manner. In some variations, the relevance score may be based on one or more factors such as word similarity between the content and the user intent (e.g., similarity in meaning, semantics, and orthography such as spelling, etc.). Different words may have different weighting factors to scale the significance of a word when assessing word similarity between content and user intent. Another factor affecting relevance score for a content candidate may be syntax structure (e.g., sentence structure). For example, a user input of “patient experienced pain in the abdomen” has a syntax structure that suggests pain in the abdomen rather than patient in the abdomen. Accordingly, diagnostic and/or treatment content relating to pain in the abdomen may have a higher relevance score than other kinds of medical content. As another example, a user input of “64 slice GE lightspeed abdomen pelvis CT protocols” has a syntax structure that is less likely to suggest 64 things, but more likely to suggest a specific machine protocol for a particular machine brand and technology (GE LIGHTSPEED computed tomography) with a specific number of slices (64) and a specific anatomical region (abdomen, pelvis). Accordingly, protocol content for these parameters may have a higher relevance score than other kinds of medical content. Other suitable factors affecting relevance score for a content candidate may include suitable rules or algorithms based on user studies, user feedback, etc. For example, content candidates including known acronyms of user intent may have lower relevance scores. As another example, colloquial or shorthand medical terminology may be “learned” by user feedback and used to adjust relevance scores appropriately. In some variations, the content scoring module 344 may include the NLP model in communication with or accessing one or more suitable medical resource databases, and the NLP model may be configured to identify content candidates and/or determine relevance scores for content candidates. Furthermore, the relevance scores for multiple content candidates may be ranked (446) (e.g., sorted according to relevance score) in order to identify medical content most likely to be associated with the predicted user intent.

In some variations, user intent and/or medical content may additionally or alternatively be predicted based at least in part on a user's previous search history and/or previous terminology (in chat conversations, note-taking, etc.). For example, for a particular user, the system may be more likely to predict user intent and/or identify medical content that is similar to the user's previous search history and/or terminology. As an illustrative example, when predicting the intent of a user input from a user who frequently searches for drug information, the intent predictor module may be more likely to predict a user intent that is drug-related. Additionally or alternatively, when determining medical content for such a user, the content scoring module may generate relevance scores that are higher for content that is drug-related. Similarly, a user's typical terminology (e.g., typically referring to a drug as “acetaminophen” instead of paracetamol or by a brand name therefor) may inform the prediction of user intent and/or determination of medical content for that user. Thus, incorporation of such user-specific data may be useful, for example, to help distinguish between multiple options for intent and/or content that otherwise are similarly likely to be appropriate (e.g., user-specific data may be a “tie-breaker” to help choose between multiple or ambiguous options).

Additionally or alternatively, user intent and/or medical content may be predicted or determined based at least in part on one or more user characteristics, such as geolocation or nationality. Accordingly, geographically-relevant data may help inform the intent predictor module and/or the content scoring module. For example, users located in (or originating from) different geographical locations may refer to the same drug in different ways. Accordingly, a user's location and/or nationality (e.g., drawn from a GPS-enabled user computing device, IP address of the user computing device, and/or user profile, etc.) may inform the prediction of user intent and/or determination of medical content for that user. As another example, users located in (or originating from) different geographical locations may use medical terminology that is characteristic of local medical association guidelines. Thus, incorporation of geographically-relevant data may be useful to help distinguish between multiple options for intent and/or content that otherwise are similarly likely to be appropriate (e.g., user-specific data may be a “tie-breaker” to help choose between multiple or ambiguous options).

As shown in FIGS. 4A and 4B, a response to the user input may be generated (450) based at least in part on the ranked relevance scores for content candidates. For example, a content candidate with the highest relevance score may be considered the most suitable content associated with the predicted user intent, and provided in a response to the user. The medical assistant system may, for example, cause the most relevant content to be displayed on the user interface of the user computing device (460, 470). The most relevant medical content may be quoted directly from the medical resource database along with a citation, and presented to the user in the user interface on the user computing device. Additionally or alternatively, in some variations, a conversation simulator 332 may be configured to receive the medical content associated with the predicted user intent (e.g., from the content scoring module 344) and generate a suitable response to the user input. For example, the conversation simulator 332 may be configured to present the medical content in a colloquial manner. Furthermore, in some variations, the generated response may include an invitation or opportunity for the user to “click through” to obtain additional related medical content. For example, the generated response may, in some instances, include only a selected portion (e.g., first paragraph, summary, etc.) of the medical content. The displayed response may be accompanied by a hyperlink that, when selected, may allow the user to access additional portions of the medical content (e.g., the displayed generated response may enable a user to “click through” to view the rest of the medical content beyond the quoted content).

In some variations, the content candidate with the highest relevance score may be selected as the most suitable content to provide to the user only if a confidence score is sufficiently above a predetermined threshold. A confidence score may be based on, for example, a statistical characteristic of the distribution of relevance scores among the content candidates (e.g., characterizing the highest relevance score as being sufficiently greater than the second-highest relevance score).

In some instances, a generated response to the user input may include multiple content candidates. For example, if two or more content candidates have relevance scores that are greater than a predetermined threshold (and/or there is insufficient confidence that any single one of the content candidates is the “best” content for responding to the user input), then multiple content candidates may be provided to the user. Upon display of the generated response with multiple content candidates (470), the user may be presented with the option to select one of the content candidates for proceeding. In some variations, a conversation simulator 332 may be configured to prompt the user to select among multiple content candidates.

Furthermore, in some instances, a generated response to the user input may include a follow-up query to the user to obtain additional information. For example, the follow-up query may seek to clarify user intent within the context of potential content candidates. In one illustration, the medical assistant system may identify generally a user intent of obtaining dosage information for a particular medication. In response, the system may generate a follow-up query to the user to clarify whether the user seeks dosage information for an adult patient or a pediatric patient. Upon receiving additional user input in response to the follow-up query, the medical assistant system may similarly parse and process the input to identify suitable medical content as described above.

In some instances, such as if no suitable content candidate if determined (e.g., no content candidate has a sufficiently high relevance score), then a generated response to the user input may omit suitable content. Instead, the generated response may include an indication that analysis of the user input was inconclusive (e.g., display a phrase such as “I don't know” or “Please rephrase your question”).

As shown in FIG. 4A, user feedback on the generated response may be received through the user interface on the user computing device (472). The feedback may include, for example, a numerical or graphical rating of the usefulness or accuracy of the generated response (e.g., rating of 1-5, rating of a discrete number of stars, etc.). As another example, the feedback may include a text-based comment (e.g., “Helpful”, “Not helpful”). Feedback may be received following display of a prompt for such feedback (e.g., “Was this helpful?”). The medical assistant system may receive such user feedback (480) and then update the NLP model or other algorithm based on the user feedback (482).

The feedback process is further illustrated in the schematic of FIG. 4C. As shown in FIG. 4C, a natural language processing model 340 may receive a user input and analyze it according to an intent predictor module 342 to predict a user intent, which may then be provided to a content scoring module 344 to determine suitable medical content. A conversation simulator 332 may incorporate the medical content and display or otherwise provide the medical content in a response to the user. A user may provide user feedback on the suitability of the generated response, and the user feedback may be provided to a learning module 334 that updates the natural language processing model and its module components. Accordingly, if the medical assistant system makes a mistake, the user can provide feedback to allow the AI system to learn from its mistakes. As such, user feedback may be used to continuously train and update the AI system. For example, user feedback may be used to adjust weighting factors for words when determining suitable medical content, and/or adjust other factors in the algorithm for determining relevance score for a content candidate. In some variations, the natural language processing model may be updated for use by all users, such that all users benefit from a modified model that is updated in view of feedback from individual users. In some variations, the natural language processing model may be updated for us by only a subset of users (e.g., only users in the same practice area as the user providing feedback).

In some variations, some kinds of user feedback may be weighted or considered more heavily than other kinds of user feedback. For example, feedback from a more experienced user (e.g., senior physician) may be treated as more influential in updating the NLP model than a less experienced user (e.g., junior physician). As another example, feedback from a user of a particular practice type regarding medical content for that practice type may be treated as more influential in updating the NLP model (e.g., feedback from a radiologist on relevance of a response to a user query regarding diagnostics using medical images may be treated as more influential).

As shown in FIG. 4A, in some variations, the medical assistant system may be configured to store at least a portion of medical content (490) in an electronic medical record associated with the patient. For example, if a user queries to the medical assistant system about suitable medication dosage for a patient, the medical assistant system may generate a response to the user query (450) including content relating to the suitable medication dosage, and then record in the patient's electronic medical record the appropriate medication dosage.

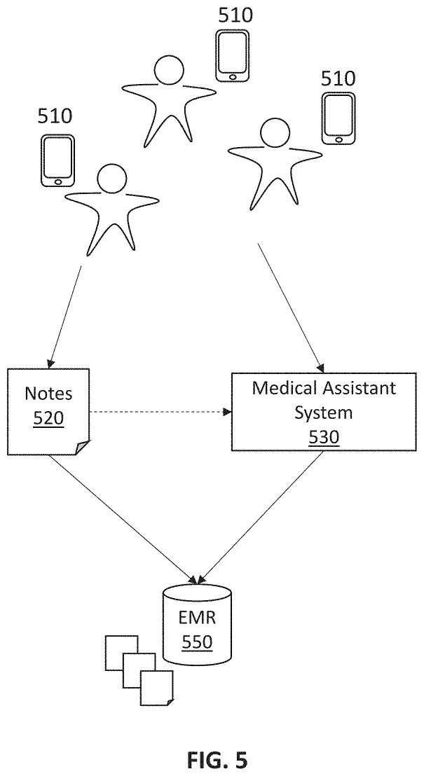

Although the operation of an AI medical assistant system is described above primarily in the context of the AI medical assistant corresponding with a single user, it should be understood that interpretation of user input and generation of suitable responses to the user input may be applied in other contexts. For example, as further described below, user input may be in the form of dialogue or group chats between different users. Furthermore, as shown in the schematic of FIG. 5, the AI medical assistant system 530 may be configured to receive user input of various kinds (e.g., within simulated conversation between the AI medical assistant and a single user 510, simulated conversation between the AI medical assistant and multiple users, conversation between multiple users on user computing devices 510 within the medical assistant application, etc.). The AI medical assistant system 530 may interpret the user input and store generated medical content in an electronic medical record 550 associated with the patient, similar to that described above.

As another example, as shown in FIG. 5, the AI medical assistant system may be configured to receive user input in the form of a note 520 (e.g., freestyle note or pre-organized case note that may be typed and/or spoken by a user) and identify suitable content for storage in an electronic medical record 550 associated with a patient. Notes may additionally or alternatively be directly stored or associated with the electronic medical record. The functionality of notes within the AI environment is further described below.

### Example GUIs

Described below are exemplary variations of graphical user interfaces (GUI) that may be implemented on a user computing device (e.g., mobile phone, tablet, or other user computer, etc.) and may be implemented in an AI environment such as that described herein.

### Chats

Generally, chat conversations may enable communication with one or more other users in the AI environment and/or with an AI medical assistant. Such communication may be used to collaborate on medical care, share medical information, and the like. In some variations, at least some of the information communicated in a chat may be stored in an electronic medical record for a patient. For example, an entire chat conversation may be stored in an electronic medical record to memorialize all content in the chat conversation. As another example, one or more selected portions of a chat conversation may be stored in an electronic medical record, where portions for storage may be identified by manual selection (e.g., user selection or tagging of individual text messages and/or attachments in a chat) and/or by the AI medical assistant monitoring content (e.g., flagging content for storage in an electronic medical record based on keywords, tags, etc.).

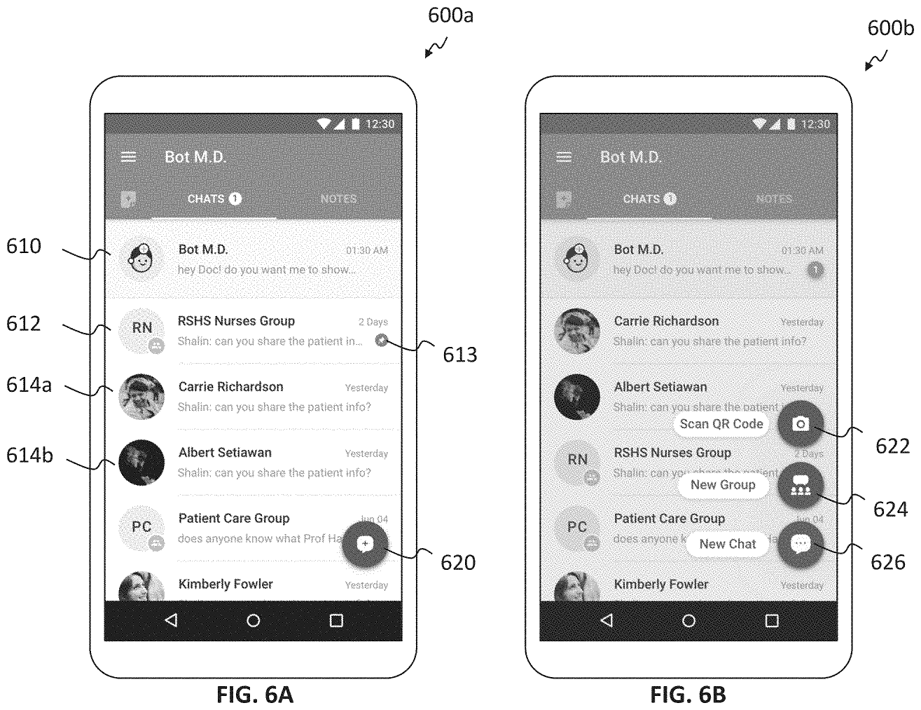

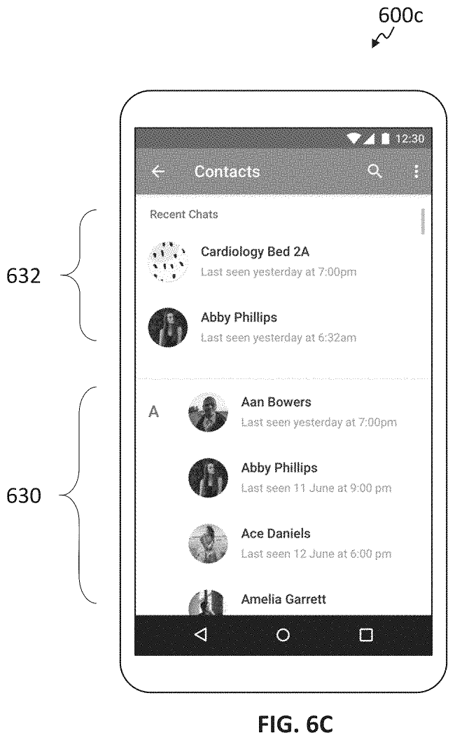

FIG. 6A is an exemplary variation of a GUI 600a displaying a list of text conversations, or chats, with various contacts within the AI environment. Different kinds of chats may be possible within the AI environment. A first chat 610 is associated with an AI medical assistant, and may be selected by a user desiring to converse with the AI medical assistant and seek medical information. A second chat 612 is associated with a group chat with multiple participating users. The multiple users within a group chat may have a shared, common characteristic (e.g., nurses working in the same department of a hospital). Additional chats 614a and 614b are individual, one-on-one chats with individual respective users. One or more of the chats may be “pinned” or fixed near the top of the list of chats, as indicated by pin icon 613, such as to enable ready access to the chat by a user. Chats may be sorted in any suitable manner, such as alphabetically by individual name or group chat name, by recency (e.g., sorted by time last viewed or modified), and/or activity (e.g., frequency of modification by chat participants), etc. In this GUI, a new chat creation icon 620 may be displayed to provide options to the user to establish new chats.

FIG. 6B is an exemplary variation of a GUI 600b displaying options for establishing new chats. For example, selection of a first chat creation icon 622 may enable a new chat with at least one other user to be created by scanning a user identifying code (e.g., QR code generated for a user) with a camera device. Selection of a second chat creation icon 624 may enable a new group chat to be created. Selection of a third chat creation icon 626 may enable a new individual chat to be created. Upon selecting the second chat creation icon 624 or third chat creation icon 626, a menu (not shown) prompting the user to select participants for the group chat may be displayed. For example, a list of contacts (similar to that shown in FIG. 6C) may be displayed to enable the user to select participants for new chats.

FIG. 6C is an exemplary variation of a GUI 600c displaying a contact list 630 including listings of other users operating within the AI environment. The contact list 630 may display contacts in any suitable order, such as alphabetically, by group association (e.g., members of a hospital), by patient assignment (e.g., contacts on the care team for a particular patient), by practice specialty, and/or frequency of contact, etc. A user may select a particular contact in the contact list 630 and initiate a chat with the selected contact and/or share files (e.g., images, videos, links, etc.) with the selected contact. In some variations, the GUI displaying the contact list 630 may additionally display a recent conversation list 632, where items in the recent conversation list 632 may be selectable by a user to shortcut to an associated recent conversation. For example, the recent conversation list 632 may be limited to n most recent conversations with other users in the AI environment, where n is a predetermined number (e.g., one, two, three, four, five, or more). As another example, the recent conversation list 632 may include all conversations last viewed (or last modified) within a predetermined preceding period of time (e.g., within the preceding day, within the preceding three days, within the preceding week, etc.). As yet another example, the recent conversation list 632 may include a set of conversations filtered by any of the above criteria.

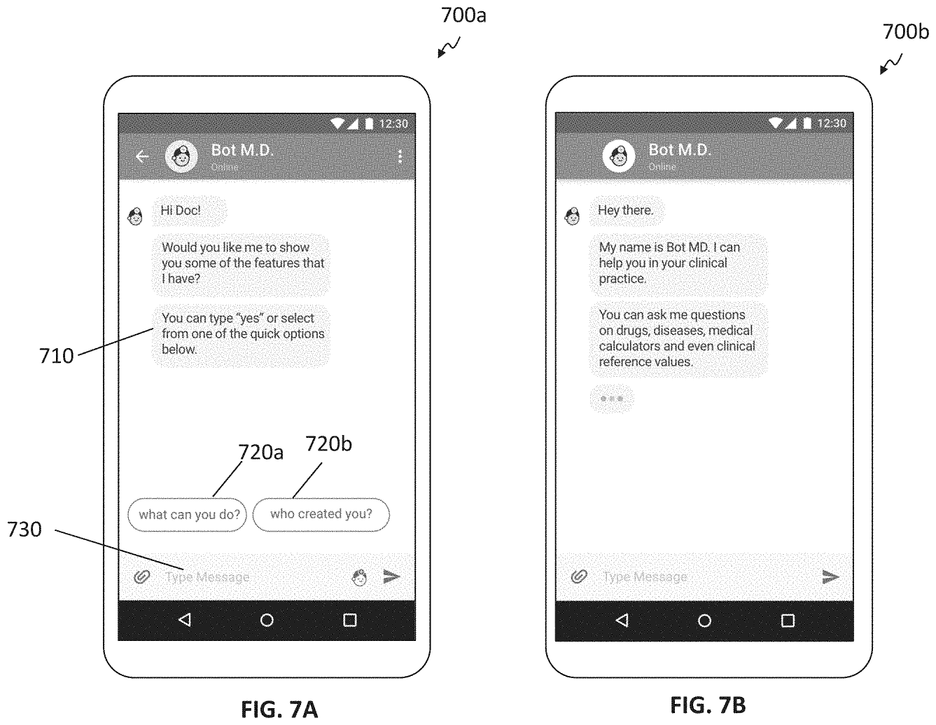

FIG. 7A is an exemplary variation of a GUI 700a displaying an individual chat with an AI medical assistant that is associated with a conversation simulator (e.g., chatbot) such that a user may interact with the AI medical assistant via text conversation. For example, the GUI 700a may display one or more messages 710 from the AI medical assistant. A user may respond to and otherwise interact with the AI medical assistant in the individual chat through GUI 700a. For example, GUI 700a may display one or more quick option text responses (e.g., 710a, 710b), which may be generated by the AI medical assistant system. For example, the one or more quick option text responses may be generated based at least in part on a decision tree or suitable matrix. The quick option text responses may be selected by a user for convenient and fast interaction with the AI medical assistant. Additionally or alternatively, a user may interact with the AI medical assistant by typing a message in the text box 730, dictating a response (which may be converted to text via a suitable speech-to-text converter).

FIG. 7B is an exemplary variation of a GUI 700b displaying an individual chat with an AI medical assistant that is associated with a conversation simulator. In GUI 700b, the AI medical assistant may prompt the user to enter suitable user inputs such as questions about drugs, diseases, medical calculators, clinical reference values, etc. As described above with reference to FIG. 7A, a user may interact with the AI medical assistant by entering a message in a text box, dictating a response, and/or selecting a quick option text response that may generated and displayed under certain circumstances.

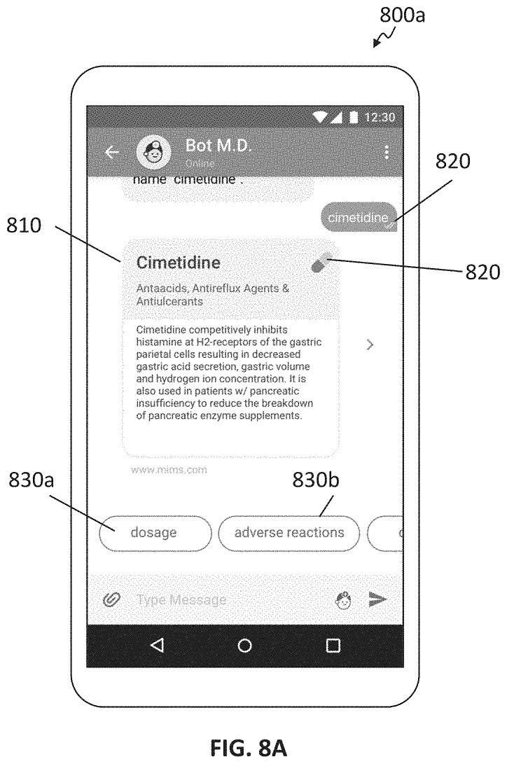

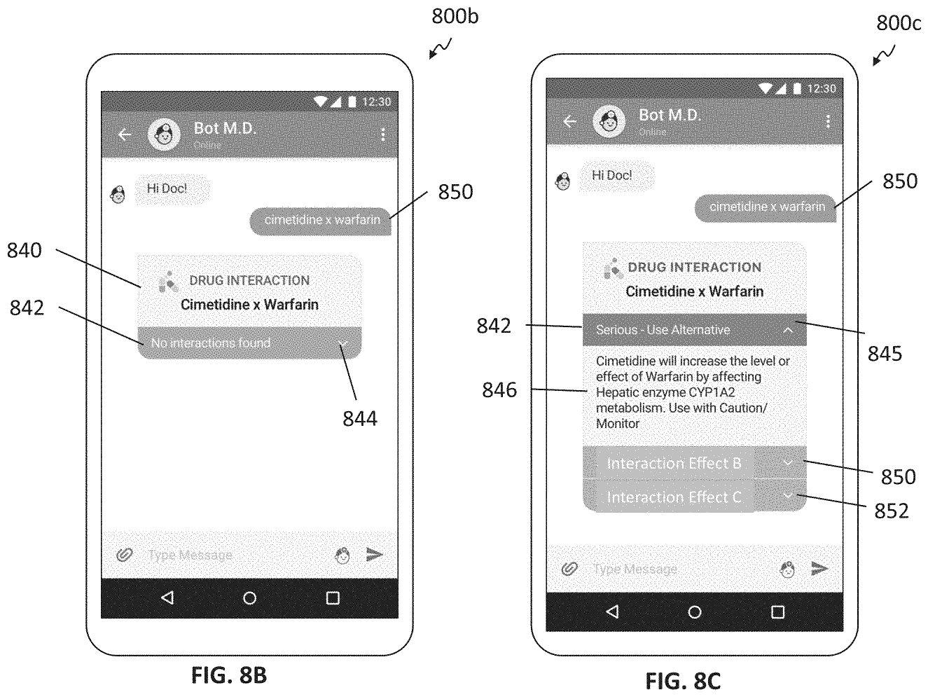

FIG. 8A is an exemplary variation of a GUI 800a displaying a medical information box 810 generated by an AI medical assistant within a conversation. For example, GUI 800a may be configured to display the drug information box 810 in response to a user input 820 including the name of a drug. The information displayed in the drug information box 810 may, for example, be generated by the AI medical assistant system interpreting the user input 820 to predict user intent and determine suitable medical content, as described above with reference to FIGS. 4A-4C.

In some variations, the drug information box 810 may include a graphical representation 820 of the drug (e.g., graphical representation of a pill capsule). The graphical representation 820 of the drug may mimic the actual appearance of the drug, and may be identified as part of the medical content associated with the user input naming the drug. As shown in FIG. 8B, the GUI 800a may further display one or more quick option text responses following the display of the drug information box 810, where each quick option text response may be selectable by a user to obtain additional information related to the drug. For example, the AI medical assistant may generate and display a first quick option text response 830a that, if selected, may result in the AI medical assistant providing dosage information for the drug. As another example, the AI medical assistant may generate and display a second quick option text response 830b that, if selected, may result in the AI medical assistant providing information regarding any adverse reactions associated with the drug. Accordingly, back-and-forth interaction between the user and the AI medical assistant may enable the user to obtain medical information as desired.

FIG. 8B is an exemplary variation of a GUI 800b displaying a drug interaction information box 840 generated by an AI medical assistant within a conversation. For example, the GUI 800b may be configured to display the drug interaction information box 840 in response to a user input 850 including an inquiry about interaction between two drugs (e.g., Cimetidine and Warfarin). The user input 850 inquiring drug interaction information may follow a predetermined format (e.g., “drug A x drug B” to obtain information about interaction between drug A and drug B) or may include a free-form text (e.g., “interaction between drug A and drug B”) that may be interpreted and processed by the AI medical assistant system. As shown in the GUI 800b, the drug interaction information box 840 may include a content summary header 842 that may summarize the drug interaction information. For example, the content summary header 842 may include text signifying risk level of the drug interaction (e.g., “No interactions found”, “Serious—Use Alternative”, etc.). In some variations, at least a portion of the drug interaction information box 840 (e.g., content summary header 842) may additionally and/or alternatively be color-coded or otherwise visually coded (e.g., with a smiley face graphic or unhappy face graphic) to indicate overall risk level of the drug interaction. For example, the content summary header 842 (and/or other portion of the drug interaction information box 840) may be colored red to indicate a serious risk of drug interaction, yellow to indicate a moderate risk of drug interaction, or green to indicate a low or no risk of drug interaction.

The drug interaction information box 840 may be expandable and collapsible to selectively show or hide content of the drug interaction information box 840. For example, as shown in FIG. 8B, the content summary header 842 (or other portion of the drug interaction information box 840) may include a dropdown arrow 844 (or text-based label such as “expand” or “open”). FIG. 8C is an exemplary variation of a GUI 800c that illustrates that when the dropdown arrow 844 is selected, the drug interaction information box 840 may expand to display additional medical content. As shown in FIG. 8C, a collapsing arrow 834 (or text-based label such as “collapse” or “close”) may be displayed, where selection of the collapsing arrow 834 may cause the drug interaction information box 840 to collapse to hide the additional medical content. Furthermore, the drug interaction information box 840 may include any suitable number of expandable and collapsible portions. For example, the drug interaction information box 840 may include second and third content summary headers 850 and 852 (and any suitable number of content summary headers). The additional content summary headers may summarize additional interaction effects, and/or any suitable content (e.g., alternative drug suggestions, etc.).

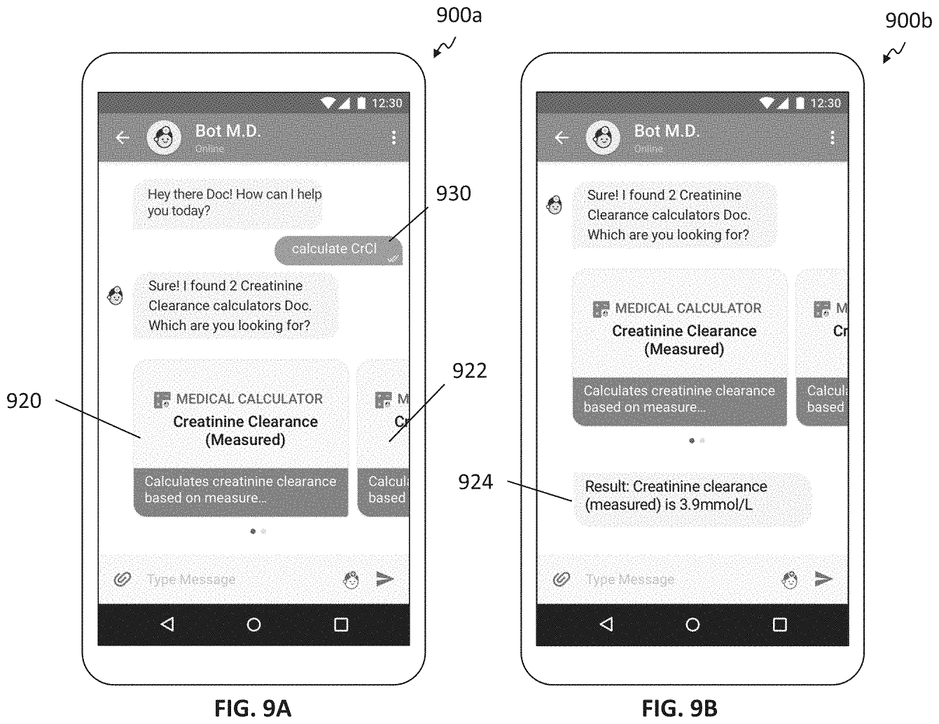

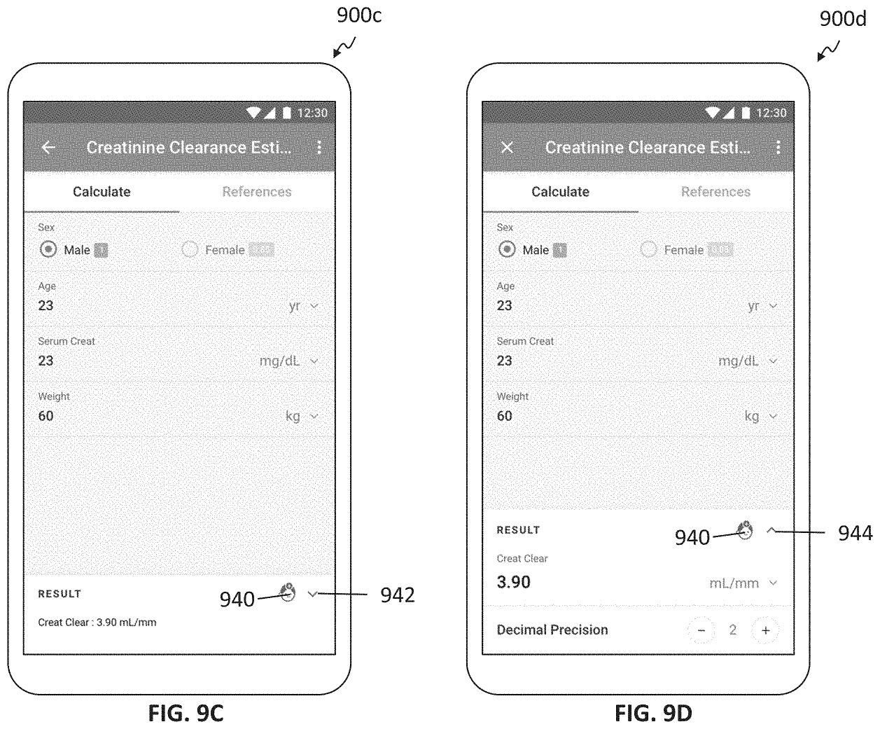

FIG. 9A is an exemplary variation of a GUI 900a displaying options for a medical calculator generated by an AI medical assistant within a conversation. For example, GUI 900a displays two calculator options in response to a user input 930 including a request for a medical calculator. The calculator options may, for example, be generated by the AI medical assistant system interpreting the user input 930 to predict user intent and determine suitable medical content, as described above with reference to FIG. 4A-4C. In the example shown in FIG. 9A, the user input 930 comprises a reference to a creatinine clearance calculator (“CrCl”) that may, for example, be used to calculate amount of creatinine that has been cleared from the blood and passed into urine, in order to evaluate kidney function. The AI medical assistant system may process the user input 930 and generate a first creatinine clearance calculator 920 that provides a measured result based at least in part on lab measurements (e.g., level of creatinine in blood samples and/or urine samples taken at predefined intervals). The AI medical assistant system may further generate a second creatinine clearance calculator 922 that provides an estimated result based at least in part on equations (e.g., level of creatinine in a blood sample entered in an equation that takes into account patient characteristics such as sex, age, weight, etc.). The GUI 900b may present both the first and second creatinine calculators as selectable options. FIG. 9B is an exemplary variation of a GUI 900b showing a medical calculator result 924 after the user has selected a medical calculator. In some variations, the AI medical assistant system may automatically pull medical information (e.g., lab test results) from an electronic medical record associated with a patient, in order to provide a patient-specific calculation result 924. Although FIGS. 9A and 9B depict creatinine clearance calculators, it should be understood that in other variations, other kinds of medical calculators may be similarly provided in response to user input.

FIG. 9C is an exemplary variation of a GUI 900c displaying a medical calculator with fields for receiving values entered by a user through the user interface, where values may be input into the medical calculator to achieve a calculated result. GUI 900c includes an AI medical assistant icon 940 that may be selectable by a user to store the calculated result, such as for future reference and/or for recording in an electronic medical record associated with a patient. In the example shown in FIG. 9C, the medical calculator is a creatinine calculator that may, for example, be used to calculate amount of creatinine that has cleared from the blood and passed into urine, in order to evaluate kidney function. The GUI 900c displays fields for sex, age, serum count, and weight, and values of these parameters may be entered in an equation to calculate creatinine clearance result, which may be displayed by the medical calculator. A user may select the AI medical assistant icon 940 to store the creatinine clearance result for future reference. Furthermore, at least a portion of GUI 900c may be expandable and collapsible to show and hide additional medical calculator content. For example, GUI 900c may display a dropdown arrow 942 (or text-based label such as “expand” or “open”). FIG. 9D is an exemplary variation of a GUI 900d that illustrates that when the dropdown arrow 942 is selected, the medical calculator may expand to display additional content (e.g., preferences) such as adjustment of decimal precision of the calculated result. As shown in FIG. 9D, a collapsing arrow 944 (or text-based label such as “collapse” or “close”) may be displayed, where selection of the collapsing arrow 944 may cause the medical calculator to collapse to hide the additional content. Although FIGS. 9C and 9D depict creatinine clearance calculators, it should be understood that in other variations, other kinds of medical calculators may be provided, and results may be similarly stored by the AI medical assistant system upon selection of the AI medical assistant icon 940. Furthermore, results may be automatically stored in an electronic medical record associated with a patient.

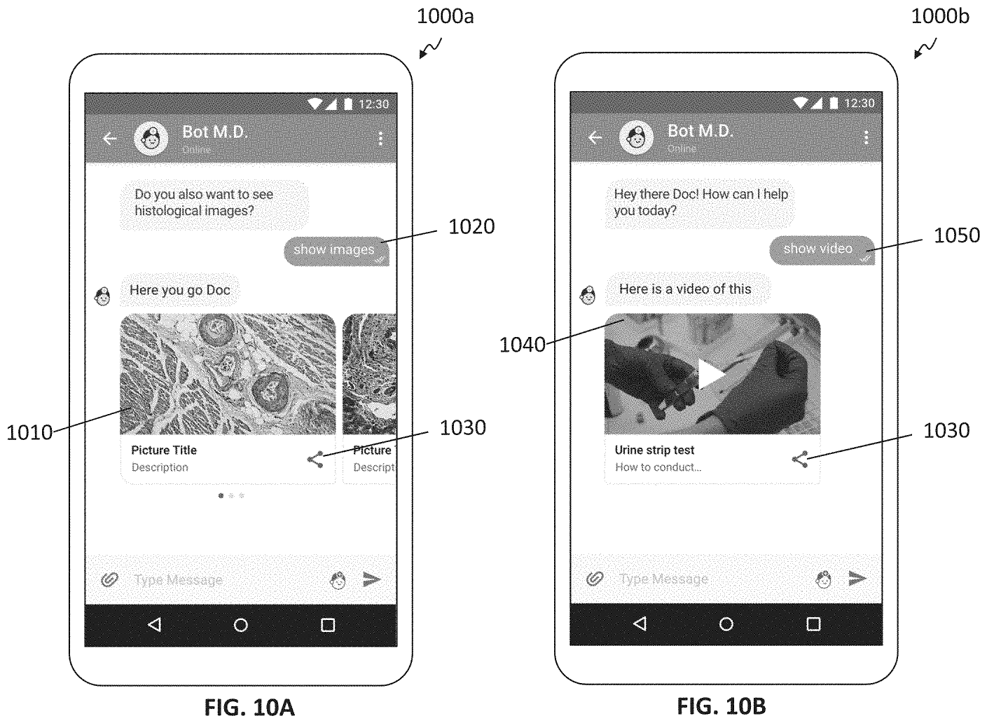

FIG. 10A is an exemplary variation of a GUI 1000a displaying at least one image 1010 within a conversation with the AI medical assistant, such as in response to a user input 1020 including a request for images. Suitable images may be include, for example images relating to a patient (e.g., “Show me patient's most recent MRI images”, “Show me Jane Doe's last chart”, etc.). The AI medical assistant system may be configured to retrieve any suitable images such as those associated with a patient (e.g., images in the patient's electronic medical record). For example, the AI medical assistant system may process a user input 1020 relating to a request to show histological images, and respond by causing display of the histological images 1010. As another example, Suitable images may include images to assist in diagnosis (e.g., “Show me a picture of eczema”). Each image may be accompanied by an image title and/or suitable description (e.g., patient name, date of image, context such as procedure or type of image, etc.). In some variations, multiple images may be displayed on the user interface. Multiple images may, for example, be arranged in a “carousel” that may be navigated by the user swiping or sliding through the images on the carousel. In some variations, an image may be further selected for sharing by selecting a share icon 1030. Selection of the share icon 1030 may, for example, prompt a menu of sharing options such as sending the image to a contact within the AI environment, emailing the image, storing the image in an electronic medical record, etc.

Other kinds of media may be presented in the user interface. For example, FIG. 10B is an exemplary variation of a GUI 1000b displaying at least one video 1040 within a conversation with the AI medical assistant, such as in response to a user input 1050 including a request for video. Suitable videos may include, for example, training videos (e.g., for a surgical procedure, for performing diagnostic procedure, etc.), video associated with a patient (e.g., from a patient consultation or treatment session) that may have been stored in an electronic medical record, etc. Similar to the images in GUI 1000a shown in FIG. 10A, each video may be accompanied by title and/or suitable description. Multiple videos may be arranged in a “carousel” that a user may navigate to view selected videos. As in GUI 1000a, a share icon 1030 may be displayed so as to be associated with a video, and selection of the share icon 1030 may prompt a menu of various sharing options to share the video with others and/or cause storage of the video in an electronic medical record, etc. Other kinds of media may include sound files (e.g., sound file of a patient with whooping cough, etc.).

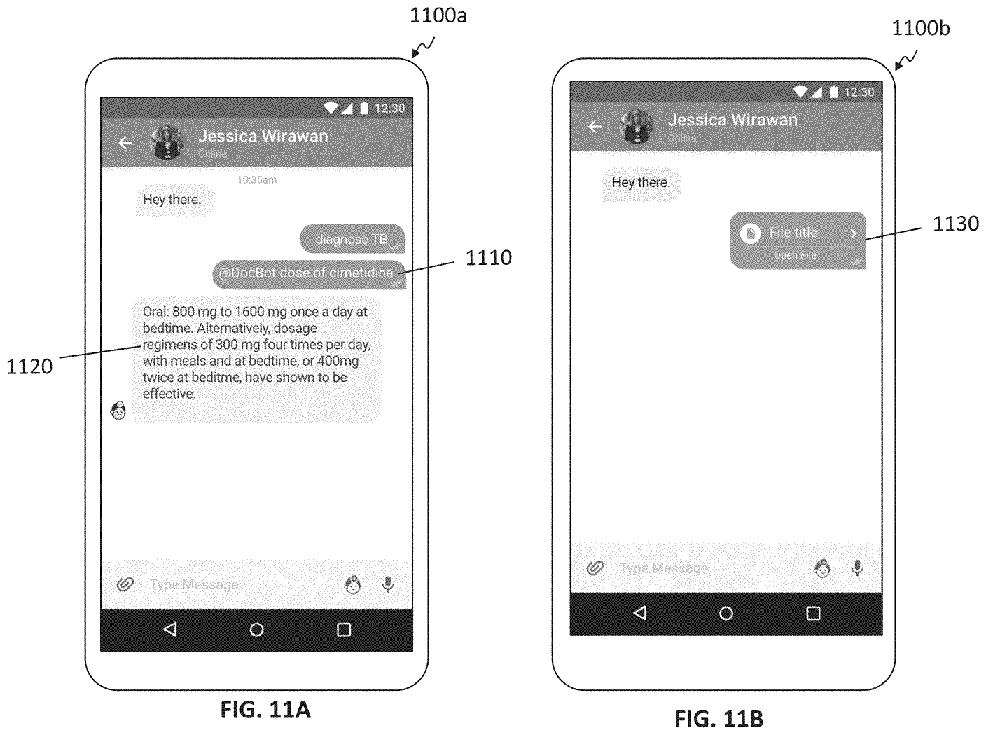

FIG. 11A is an exemplary variation of a GUI 1100a displaying a response by the AI medical assistant in response to a user input 1110 within a conversation with one or more other users (e.g., individual chat, group chat). For example, user input 110 may include a callout or tag (e.g., “@ [assistant name]”) accompanying a particular user query to invite a response from the AI medical assistant though the user is not directly in a one-on-one conversation with the AI medical assistant. Similar to processes described above, when called into a conversation, the AI medical assistant may process the user input (predict user intent, determine medical content, etc.) and cause display of a generated response 1120 to the user input. Accordingly, the response from the AI medical assistant may be viewed by both the user and any other users in the conversation.

FIG. 11B is an exemplary variation of a GUI 1100b displaying file sharing with another user within a conversation in the AI environment. For example, message 1130 to another user may indicate a particular file (e.g., notes document, image, video, website link, etc.) that is being sent to another user. Although GUI 1100b depicts file sharing with another individual user, it should be understood that similarly any suitable file can be shared through a variation of GUI 1100b to multiple users, such as in a group chat conversation.

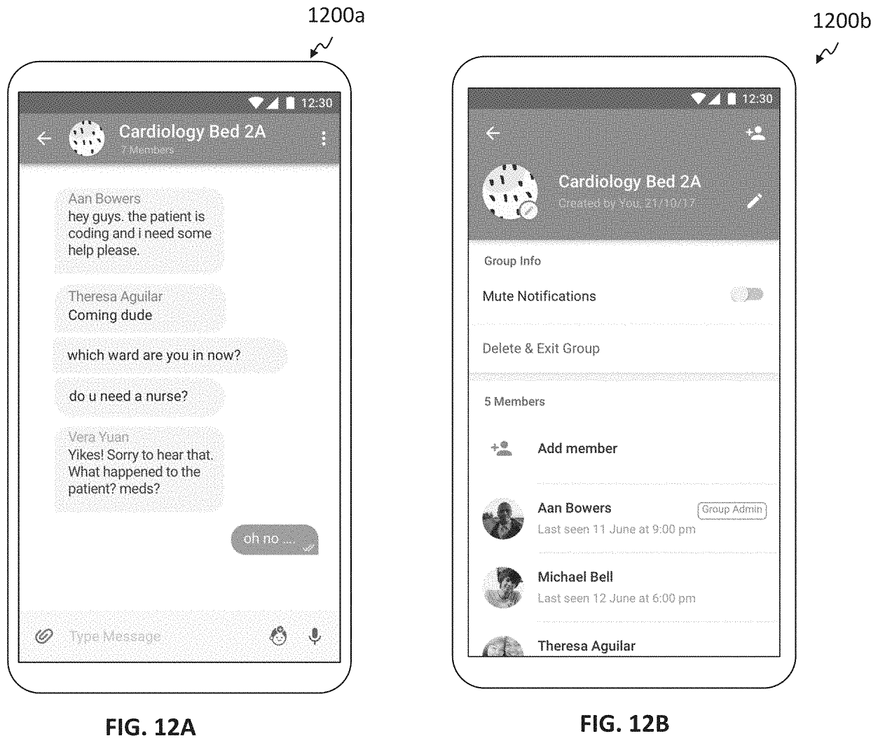

FIG. 12A is an exemplary variation of a GUI 1200a displaying a group chat conversation. As described above, a group chat may include any suitable set of participating users desiring to communicate with each other (e.g., on respective user computing devices). For example, multiple users in a group chat may include caretakers (e.g., physicians, nurses, etc.) for a particular patient, or caretakers operating within the same area such as the same hospital department. As another example, multiple users in a group chat may share common practice areas or interest areas (e.g., all users in a group chat may be radiologists, or all users in a group may be anesthesiologists). Accordingly, in some variations, members of a group chat may collaborate for medical treatment of a patient through the AI environment. FIG. 12B is an exemplary variation of a GUI 1200b displaying options for configuring a group chat. For example, GUI 1200b may be navigable to add or remove members of the group chat, designate one or more users as a group administrator (e.g., who may authorize edits to membership of the group chat and/or configure settings of the group chat), edit the name of the group chat, and/or adjust notification settings.

### Notes

Generally, notes may be entered by a user to generate a record of medical information or any suitable information that may be desirable to have for future reference. A note may, for example, include clinical information relating to a patient (e.g., case note) or any suitable comments that a user may wish to record. As further described below, a note may include attachments such as media (e.g., image files, video files, sound files, etc.) or hyperlinks to other content. Notes may be shared with one or more other users and/or stored in an electronic medical record.

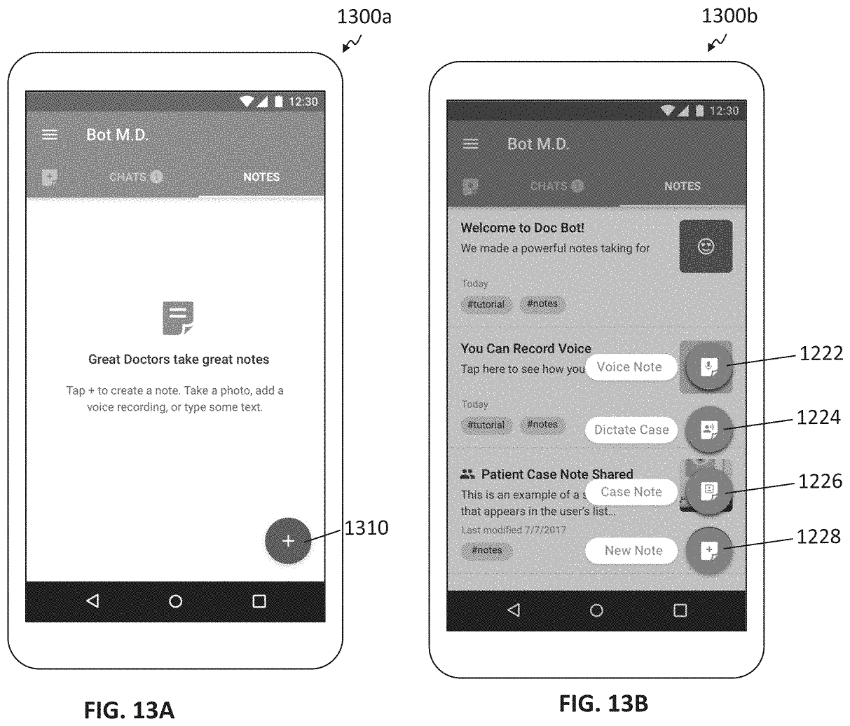

FIG. 13A is an exemplary variation of a GUI 1300a configured to display notes that are accessible to a user. For example, a list of notes may be displayed in GUI 1300a and selectable for viewing (if a text-based note) and/or listening (if an audio-based note). Notes listed in GUI 1300a may include notes entered by a user and/or notes received from other users or pulled from an electronic medical record, etc. In the GUI 1300a, creation of a new note may be initiated by selecting a note menu icon 1310, which may prompt display of a menu of note-taking options.

FIG. 13B is an exemplary variation of a GUI 1300b displaying a menu of note-taking options. For example, new note icon 1222 is selectable by a user to initiate a freestyle, dictated note that may be captured and recorded by a microphone device that is on or in communication with a user computing device. New note icon 1224 is selectable by a user to initiate a dictated case note that may be captured and recorded by a microphone device, and where the note content may be automatically formatted to follow a predetermined template for a case note (e.g., associated with a patient). New note icon 1226 is selectable by a user to initiate a typed case note that may be entered via a keyboard interface, and where the note content may be formatted to follow a predetermined template for a case note. New note icon 1228 may be selectable by a user to initiate a freestyle typed note that may be entered via a keyboard interface. Examples of these note taking options are described in further detail below.

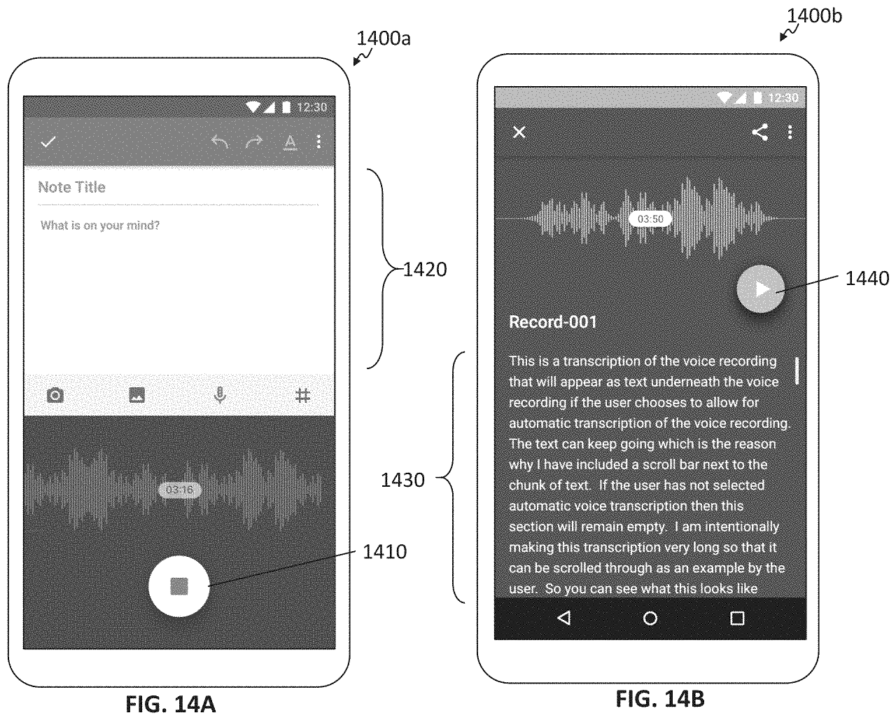

FIG. 14A is an exemplary variation of a GUI 1400a displaying a freestyle voice note-taking interface. To initiate voice note-taking, a user may select a recording start/stop icon 1410 and begin dictating contents for the note. The GUI 1400a may additionally enable entering text-based information through a keyboard (e.g., note title). In some variations, a suitable speech-to-text converter may transcribe the dictated voice note, such as while the user is speaking and/or after the user has finished speaking. The transcribed contents of the voice note may appear in the transcription region 1420. In some variations, the transcription may be edited and/or supplemented through the GUI 1400a, such as by entering text on a keyboard interface. After the voice note is recorded, the voice note and/or its transcription may be saved and stored in an electronic medical record for future access (e.g., by a user and/or by an AI medical assistant, for viewing and/or for sharing).

FIG. 14B is an exemplary variation of a GUI 1400b displaying a transcribed freestyle voice note. A transcription of the voice note may be displayed in transcription region 1430. In some variations, the transcription may be edited and/or supplemented through the GUI 1400b, such as by entering text on a keyboard interface. Audio playback of a voice note may be initiated by selecting playback start/stop icon 1440.

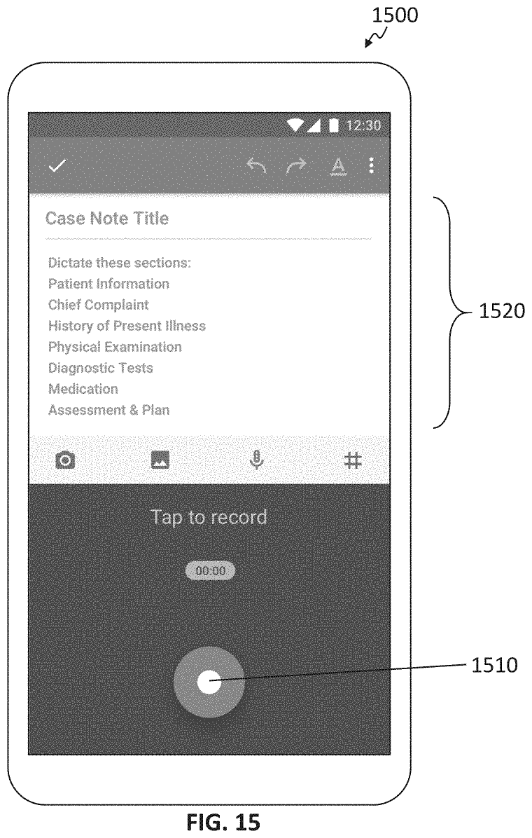

FIG. 15 is an exemplary variation of a GUI 1500 displaying a dictated voice case note-taking interface. To initiate dictated voice case note-taking, a user may select a recording start/stop icon 1510 and begin dictating contents for the case note. In creating a dictated voice case note, a user may dictate predetermined section titles according to a case note template. For example, a user may dictate sections such as “Patient Information”, “Chief Compliant”, “History of Present Illness”, “Physical Examination”, “Diagnostic Tests”, “Medication” “Assessment & Plan”, or any suitable section titles. An AI medical assistant or other suitable processor may recognize dictation of section titles and automatically partition one or more segments of the dictation following each dictated section title, to be formatted into the dictated case note sections accordingly. In some variations, a suitable speech-to-text converted may transcribe the dictated case note, such as while the user is speaking and/or after the user has finished speaking. The transcribed contents of the voice case note may appear in the transcription region 1520. In some variations, the transcription may be edited and/or supplemented through the GUI 1500 or other suitable GUI (e.g., similar to GUI 1400b described above), such as by entering text on a keyboard interface. After the voice case note is recorded, the voice note and/or its transcription may be saved and stored in an electronic medical record for future access (e.g., by a user and/or by an AI medical assistant, for viewing and/or for sharing).

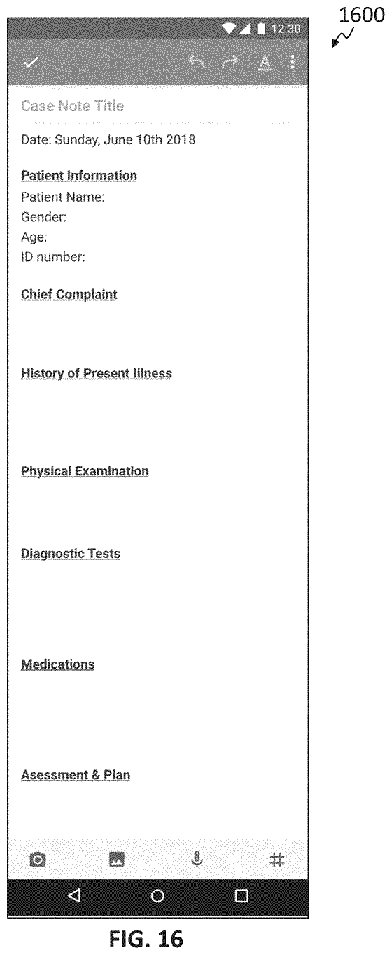

FIG. 16 is an exemplary variation of a GUI 1600 displaying an interface for completing a typed case note, such as via a keyboard interface. Similar to the voice case note option described above, the typed case note may include predetermined section titles according to a case note template. A user may use a keyboard interface on or connected to the user computing device to populate the predetermined sections with clinical information for a patient. The typed case note may include a single text box, and/or may include predetermined text boxes of fields that may be individually populated. After the typed case note is at least partially populated, the typed case note may be saved and stored in an electronic medical record for future access (e.g., by a user and/or by an AI medical assistant, for viewing and/or for sharing).

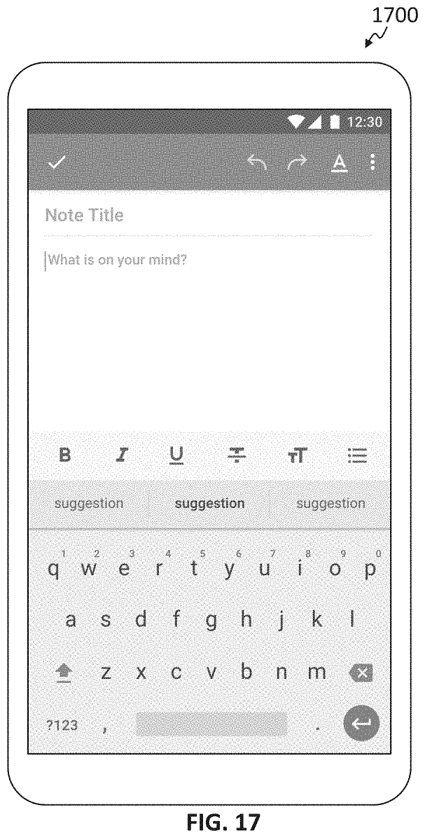

FIG. 17 is an exemplary variation of a GUI 1700 displaying an interface for completing a freestyle typed note, such as via a keyboard interface. In contrast to a case note with prepopulated section titles, the freestyle typed note may omit all section titles. For example, the freestyle typed note may be formatted similar to a single text box. After the freestyle typed note is completed, the typed note may be saved and stored in an electronic medical record for future access (e.g., by a user and/or by an AI medical assistant, for viewing and/or for sharing).

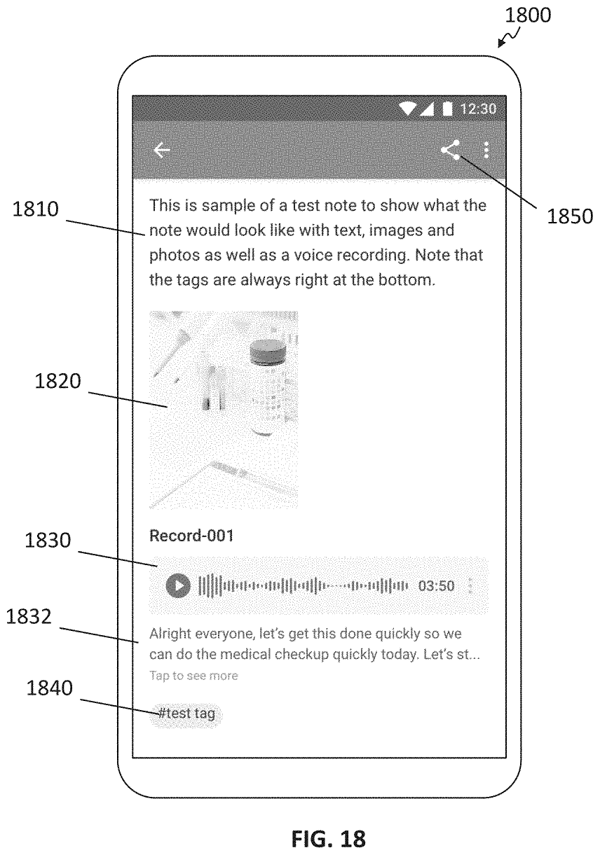

In some variations, a GUI may enable note-taking that combines various features (e.g., dictated note-taking, typed note-taking, etc.) in one “combination” note. FIG. 18 is an exemplary variation of a GUI 1800 displaying an exemplary combination note. The combination note may include a text portion 1810 entered with a keyboard interface, and a voice portion 1830 entered by dictation. A transcription of the voice portion 1830 may appear in a transcription region 1832. As with other notes described above, after the combination note is entered, the combination note may be saved and stored in an electronic medical record for future access (e.g., by a user and/or by an AI medical assistant, for viewing and/or for sharing).

In some GUI variations, one or more attachments may be entered to a note and stored therewith. For example, as shown in FIG. 18 in GUI 1800, at least one image 1820 may be entered into the note (e.g., by selection from a submenu, etc.). Similarly, video or other suitable media, links to other notes, etc. may be entered into the note. Any of the above-described notes (freestyle note or case note, dictated or typed, etc.) may include any suitable attachments entered into the note and stored therewith.

Furthermore, in some GUI variations, one or more tags (e.g., hashtags) may be entered and associated with a note. For example, thematic tags such as “diagnostics”, “images”, “drugs”, “treatment”, etc. may be associated with a note. Such tags may enable notes with common features to be quickly retrieved and viewed together, facilitate organization of notes, etc. Any of the above-described notes (freestyle note or case note, dictated or typed, etc.) may have any suitable tags associated therewith.

### Other GUI Features

In some variations, chat GUIs and/or note GUIs such as those described above may require network connectivity to the AI environment (or other server, etc.) to enable a user to access medical information, such as chat and/or note creation or storing functionalities described herein. However, in some variations, at least some medical information may be accessible for offline access. For example, at least some selected medical content may be downloaded to a local memory device on a user computing device. Accordingly, an AI medical assistant may be able to search within the downloaded medical content even when the user's computing device is offline, and provide seamless user interaction with the AI medical assistant system within the scope of the downloaded medical content. In some variations, certain medical content may be explicitly designated by a user for downloading (e.g., manual selection of listed content, through commands with the AI medical assistant, etc.). Additionally or alternatively, certain medical content may be automatically or semi-automatically designated for downloading based on user characteristics. For example, if a user's profile within the AI environment indicates that the user is an anesthesiologist, medical content relating to dosage requirements for certain kinds of anesthesia may be automatically designated for downloading to the user's computing device.

As described above, medical information may be easily shared among users within the AI environment. Such medical information may, in some instances, include sensitive information. In some variations, it may be desirable to facilitate “temporary sharing” of such content, such that shared content may be viewed by a recipient for a limited period of time before the shared content is deleted or otherwise removed from access by the recipient. For example, shared content may be selectively designated for deletion after a predetermined time such as 10 seconds, 30 seconds, a minute, 10 minutes, or any suitable period of time. The predetermined time period may begin when the shared content is sent, when the shared content is received by a recipient, when the shared content is first viewed, when the shared content is viewed by the last person in a group chat, or any suitable time. Furthermore, some GUI variations may enable “remote deletion” on command, such that a sender of shared content or other user may designate selected shared content for deletion. In some variations, shared content may additionally or alternatively be protected by other security schemes, such as passwords or passcodes, or geolocation-limited access (e.g., a recipient may only view shared content relating to a patient when he or she is located within a hospital where the patient is located).

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

1. A method for processing information, comprising:
causing display of a user interface on a user computing device, wherein the user interface comprises a conversation simulator;
receiving user input from at least one user provided through the user interface, the user input relating to medical treatment of a patient;
predicting a user intent based on at least one keyword in the user input;
determining medical content based on the user intent and at least one candidate medical content associated with the user intent;
automatically generating a response to the user input based on the user intent and medical content; and
causing display of the generated response to the user input on the user interface through the conversation simulator.

2. The method of claim 1, wherein the user input comprises text-based user input.

3. The method of claim 1, wherein the user input comprises auditory user input.

4. The method of claim 1, wherein the user input comprises dialogue between two or more users within the user interface, wherein the method further comprises monitoring the dialogue between the two or more users.

5. The method of claim 4, wherein monitoring the dialogue between the two or more users comprises identifying at least a first keyword associated with user intent and a second keyword associated with medical content.

6. The method of claim 5, further comprising storing at least a portion of the medical content in an electronic medical record associated with the patient.

7. The method of claim 1, wherein the conversation simulator is associated with a natural language processing model.

8. The method of claim 7, wherein predicting a user intent comprises determining at least one synonym of the at least one keyword.

9. The method of claim 8, wherein determining medical content comprises mapping at least one of the keyword and synonym to at least one medical content candidate according to a model.

10. The method of claim 9, wherein determining medical content comprises determining a relevance score for each medical content candidate and comparing the relevance scores.

11. The method of claim 10, further comprising receiving user feedback relating to the quality of the generated response.

12. The method of claim 11, further comprising modifying the model based at least in part on the user feedback.

13. The method of claim 1, further comprising storing at least a portion of the medical content in an electronic medical record associated with the patient.

14. The method of claim 1, further comprising receiving a user-entered note associated with the patient and storing at least a portion of the user-entered note in an electronic medical record associated with the patient.

15. A system for processing information, comprising:
one or more processors configured to:
cause display of a user interface on a user computing device, wherein the user interface comprises a conversation simulator;
receive user input from at least one user through the user interface, the user input relating to medical treatment of a patient;
predict a user intent based on at least one keyword in the user input;
determine medical content based on the user intent and at least one candidate medical content associated with the user intent;
automatically generate a response to the user input based on the user intent and medical content; and
cause display of the generated response to the at least one user on the user interface through the conversation simulator.

16. The system of claim 15, wherein the one or more processors is configured to predict a user intent at least in part by determining at least one synonym of the at least one keyword.

17. The system of claim 16, wherein the one or more processors is configured to determine medical content at least in part by mapping at least one of the keyword and the synonym to at least one medical content candidate according to a model.

18. The system of claim 17, wherein the one or more processors is configured to determine medical content at least in part by determining a relevance score for each medical content candidate and comparing the relevance scores.

19. The system of claim 15, wherein the one or more processors is configured to cause storing at least a portion of the medical content in an electronic medical record associated with the patient.

20. The system of claim 15, wherein the one or more processors is configured to store at least a portion of a user-entered note associated with the patient in an electronic medical record associated with the patient.
